(19) United States
(12) Patent Application Publication
(10) Pub. No.: US 2016/0264655 A1
(43) Pub. Date: Sep. 15, 2016

(54) Treating cancers with drugs targeting CREB3L1

(71) Applicant: Board of Regents, The University of Texas System, Dallas, TX (US)

(72) Inventors: Jin Ye, Dallas, TX (US); Bray Denard, Dallas, TX (US)

(73) Assignee: Board of Regents, The University of Texas System, Dallas, TX (US)

(21) Appl. No.: 15/162,566

(22) Filed: May 23, 2016

Related U.S. Application Data
(63) Continuation of application No. PCT/US14/66819, filed on Nov. 21, 2014.
(60) Provisional application No. 61/908,034, filed on Nov. 22, 2013.

(51) Int. Cl. C07K 16/18; G01N 33/574; A61K 31/704

(52) U.S. Cl. CPC C07K 16/18; A61K 31/704; G01N 33/57492; C07K 2317/34; C07K 2317/33

(57) ABSTRACT

CREB3L1 is a biomarker for identifying cancer cells sensitive to doxorubicin (daunomycin). The biomarker is used in diagnostics and screening; for example, to indicate suitability of doxorubicin chemotherapy and for pharmacological screens for agents that act through the same pathway

This application claims priority to U.S. 61/908,034, filed Nov. 22, 2013.

This invention was made with government support under grants 5R01-AI-090119-02 and HL20948 awarded by the National Institutes of Health. The government has certain rights in this invention.

### INTRODUCTION

Doxorubicin (Adriamycin®) is used widely to treat diverse types of cancer, yet its effectiveness is hampered by the existence of drug-resistant cancer cells. The reason for drug resistance is unclear mainly because the mechanism through which doxorubicin inhibits proliferation of cancer cells is not completely understood. Doxorubicin has been proposed to exert its cytostatic action through intercalation into DNA and production of free radicals (1). However, these mechanisms are unlikely to be clinically relevant as the concentration of doxorubicin required to produce these effects is much higher than that achievable in patients (1). Inhibition of topoisomerase II by doxorubicin at clinically achievable concentrations leads to DNA breaks, but a consistent relationship between DNA strand breaks and the cytostatic action of the drug has not been demonstrated (1). Thus, the mechanism through which doxorubicin inhibits cell proliferation remains unclear.

In addition to blocking cell proliferation, doxorubicin induces renal fibrosis in mice by stimulating production of collagen (2). The dual ability of doxorubicin to block cell proliferation and to induce collagen expression caught our attention inasmuch as we recently showed that both responses can be activated by a transcription factor called cAMP response element-binding protein 3-like 1 (CREB3L1, also known as OASIS) (3). CREB3L1 belongs to a family of transcription factors synthesized as transmembrane precursors (4) and activated through a process designated as Regulated Intramembrane Proteolysis (RIP) (5). The transcription factor domain of CREB3L1 is located in the NH2-terminal 374-amino acids that project into the cytosol, The COOH— terminal domain of 124 amino acids projects into the lumen of the endoplasmic reticulum (ER). Viral infection triggers the RIP of CREB3L1, which undergoes two sequential cleavages mediated by Site-1 protease (S1P) and Site-2 protease (S2P) (3). The S1P-catalyzed cleavage at the luminal side is a prerequisite for the S2P-catalyzed intramembrane cleavage that releases the NH2-terminal domain of the protein from membranes, allowing it to drive transcription of genes that suppress cell proliferation such as p21 (3). Nuclear CREB3L1 also activates genes required for assembly of the collagen matrix, including collagen 1a1 (3). Considering these dual activities we hypothesized that doxorubicin might function by stimulating proteolytic activation of CREB3L1.

Here we report that doxorubicin stimulates de novo synthesis of ceramide, which in turn activates CREB3L1, a transcription factor synthesized as a membrane-bound precursor, and disclose that measurement of CREB3L1 expression is a useful biomarker in identifying cancer cells sensitive to doxorubicin.

### SUMMARY OF THE INVENTION

In one aspect the invention provides a method of determining a suitable chemotherapy for a cancer patient determined to be in need thereof, the method comprising the step(s) of: detecting CREB3L1 expression of cancer cells of the patient in a sample obtained from the patient, wherein the expression indicates suitability for the patient of doxorubicin chemotherapy, wherein the detecting step comprises immunodetection of CREB3L1 with an antibody that specifically binds an epitope within residues 7-41 of CREB3L1.

In embodiments:
the method doses doxorubicin chemotherapy for the cancer patient, wherein the expression indicates suitability for the patient of high dosage doxorubicin chemotherapy, as opposed to a conventional lower standard dosage that would otherwise be indicated;
the method further comprises the step of isolating or obtaining the sample as a biopsy from the patient;
the method further comprises the steps: translating the CREB3L1 expression to an indication of doxorubicin chemotherapy suitability for the patient; and recording the indication;
the method further comprises the step(s) of: (i) providing for the patient information indicating said suitability of a CREB3L1-mediated chemotherapy; (ii) providing for the patient a prescription for the CREB3L1-mediated chemotherapy; and/or (iii) administering to the patient the CREB3L1-mediated chemotherapy; and/or
the method consists of or consists essentially of the recited step(s)

In another aspect the invention provides a method of detecting CREB3L1 comprising immunodetecting the CREB3L1 with an antibody that specifically binds an epitope within residues 7-41 of CREB3L1.

In embodiments,
the method is a drug screen and comprises determining a candidate anti-cancer drug activates CREB3L1, and determining that the drug inhibits growth of a CREB3L1 expressing cancer cell; and/or
the method comprises immunohistochemistry (IHC) detection of the CREB3L1 in a tumor sample.

In another aspect the invention provides an isolated antibody that specifically binds an epitope within residues 7-41 of CREB3L1, or a monoclonal antibody that specifically binds an epitope within residues 7-41 of CREB3L1.

In embodiments, the antibody is a mouse monoclonal antibody.

The invention provides all combinations of particular embodiments of these aspects.

### DETAILED DESCRIPTION OF PARTICULAR EMBODIMENTS

We disclose that CREB3L1 is a novel tumor suppressor. We have found that doxorubicin inhibits tumor cell proliferation by activating CREB3L1. We disclose that CREB3L1 is a novel drug target to treat cancer and a novel biomarker to identify cancer cells that are sensitive to CREB3L1-mediated chemotherapy treatment. We disclose that measuring CREB3L1 expression in cancer cells is useful in identifying cancer patients who are most likely to benefit from such chemotherapy treatment.

Doxorubicin is used widely to treat diverse types of cancer, yet its effectiveness is hampered by the existence of drug-resistant cancer cells. The reason for drug resistance is unclear mainly because the mechanism through which doxorubicin inhibits proliferation of cancer cells is not completely understood. Our observation that doxorubicin inhibits proliferation of cancer cells through CREB3L1 activation indicates that measurement of CREB3L1 expression is a useful biomarker in identifying cancer cells sensitive to doxombicin. By restricting application of doxombicin only to the patients that are most likely to benefit from the drug treatment (i.e. those whose cancer cells express CREB3L1) our methods will reduce the number of the dmg-resistant cases.

Our data indicate that CREB3L1 is specifically required for doxorubicin but not for other chemotherapeutic reagents such as etoposide, bleomycin and paclitaxel to block cell proliferation; and doxorubicin-induced ceramide synthesis but not DNA breaks is required to stimulate cleavage of CREB3L1.

The invention provides a biomarker for identifying cancer cells sensitive to doxorubicin (daunomycin), and methods of using the biomarker in diagnostics and screening, e.g. to indicate suitability of doxorubicin chemotherapy and for pharmacological screens for agents that act through the same pathway.

Our claims rely on, but do not preempt our finding that CREB3L1 is a novel tumor suppressor; rather our claims are limited to a particular application of that finding, tailoring doxorubicin chemotherapy to a particular class of cancer patients whose cancer cells express CRB3L1 and are particularly sensitive and responsive to doxorubicin chemotherapy.

In one aspect the invention provides methods of determining a suitable chemotherapy for a cancer patient determined to be in need thereof, comprising the step of detecting CREB3L1 expression of cancer cells of the patient in a sample or biopsy obtained from the patient, wherein the expression indicates suitability for the patient of doxorubicin chemotherapy.

In another aspect the invention provides methods of determining chemotherapy for a cancer patient determined to be in need thereof, comprising step(s): (i) detecting CREB3L1 expression of cancer cells of the patient in a sample (biopsy) obtained from the patient; (ii) translating the CREB3L1 expression to an indication of doxorubicin chemotherapy suitability for the patient; and (iii) recording the indication.

These aspects would equivalently indicate the suitability of alternative CREB3L1-mediated chemotherapies that similarly work by activating de novo ceramide synthesis to inhibit cell proliferation.

These aspects may also be expressed as a method of dosing doxorubicin chemotherapy for a cancer patient determined to be in need thereof comprising the recitec step(s).

The invention provides all combinations of particular embodiments of these aspects, wherein:

(a) the detecting step comprises immunodetection of CREB3L1;

(b) the expression indicates suitability for the patient of high dosage doxorubicin chemotherapy, or the indication of doxorubicin chemotherapy suitability indicates suitability of high dosage doxorubicin chemotherapy, as opposed to lower, conventional dosage chemotherapy that would be indicated in the absence of CREB3L1 expression; hence the method may comprise converting a first, conventional or default dosage, to a second elevated dosage, higher than the first, based upon the CREB3L1 expression;

(c) the method further comprises the step of recording an indication of the suitability for the patient of doxorubicin chemotherapy;

(d) the method further comprises the step of isolating (obtaining) the sample (biopsy) from the patient; and/or

(e) the method further comprises step(s):

(i) providing for the patient information indicating said suitability of a CREB3L1-mediated chemotherapy; (ii) providing for the patient a prescription for the CREB3L1-mediated chemotherapy; or (iii) administering to the patient the CREB3L1-mediated chemotherapy.

For example, in various embodiments, particular embodiments (a) and (b), or (a), (b), and (c), or (a), (b), (c) and (d), (a) and (c), etc., may be combined.

In some embodiments the method consists of the recited step(s), or consists essentially of the recited step(s), which means that the method does not include other steps which materially contribute to the stated outcome, e.g. the indication.

In another aspect the invention provides methods of drug screening, comprising step(s): determining a candidate anti-cancer drug activates CREB3L1, wherein the determining step optionally comprises immunodetection of CREB3L1, and wherein the method optionally further comprises the step of determining that that the drug inhibits growth of a CREB3L1 expressing cancer cell.

The correlation between CERB3L1 expression and doxorubicin sensitivity is confirmed across 45 different human breast cancer cell lines, and demonstrated with human breast cancer cells in vivo, in a xenograft model. In particular, the relationship between CREB3L1 expression and doxorubicin sensitivity is confirmed in a study of 45 lines of breast cancer cells from ATCC Breast Cancer Cell Panel. The doxorubicin IC₅₀ value in each line of the cells plotted against the amount of CREB3L1 mRNA expressed in these cells for the entire collection of the cell panel confirms a negative correlation between the two parameters. The relationship between CERB3L1 expression and doxorubicin sensitivity is also confirmed in vivo by injection of MCF7 with or without overexpression of CREB3L1 into athymic nude mice. These mice are then treated with different doses of doxorubicin, confirming that the dose of doxorubicin required to shrink tumors produced from MCF7 cells overexpressing CREB3L1 are lower than those required to shrink tumors generated from those without the overexpression.

### EXAMPLES

### Doxorubicin Blocks Proliferation of Cancer Cells Through Proteolytic Activation of CREB3L1

We fractionated human hepatoma Huh7 cells (6) into membrane and nuclear fractions, and used an antibody reacting against the NH2-terminal domain of CREB3L1 (3) to examine the cleavage of CREB3L1 through immunoblot analysis. In the absence of doxorubicin, CREB3L1 existed as the full length precursor (˜80 kDa) in membranes and the cleaved nuclear form of CREB3L1 (˜55 kDa) was barely detectable. Treatment with doxorubicin markedly raised the amount of the nuclear form of CREB3L1. The amount of membrane protein calnexin and nuclear protein lysine-specific demethylase 1 (LSD1) was not altered by doxorubicin treatment. To determine whether doxorubicin-stimulated cleavage of CREB3L1 was catalyzed by S1P and S2P, we analyzed the cleavage in mutant Chinese Hamster Ovary (CHO) cells deficient in S1P or S2P (7, 8). In wild type CHO cells, doxorubicin stimulated cleavage of CREB3L1 to produce the nuclear form. In contrast, doxorubicin failed to produce the nuclear form of CREB3L1 in mutant cells deficient in either S1P or S2P. In wild type CHO cells, we also detected in the membrane fraction a cleaved fragment with a molecular weight similar to that of the nuclear form. This fragment was absent in cells deficient in S1P but dramatically elevated in cells deficient in S2P. These findings indicate that this membrane-bound fragment is the intermediate form of CREB3L1 that was cleaved by S1P but not by S2P. Similar cleavage intermediates were observed in earlier studies of SREBP-2 and ATF6, two prototypes of RIP substrates, in mutant CHO cells deficient in S2P (8, 9).

In Huh7 cells transfected with a control shRNA (Huh7-shControl), doxorubicin induced the expression of collagen 1a1 and p21, both of which were shown to be direct targets of CREB3L1 (3, 10). In Huh7 cells stably transfected with a shRNA targeting CREB3L1 (Huh7-shCREB3L1) in which expression of CREB3L1 was drastically reduced (3), Induction of these genes was markedly blunted. Inasmuch as CREB3L1 was required for doxorubicin to induce expression of p21, a well-characterized inhibitor of the cell cycle (11), we determined whether CREB3L1 was also required for the drug to inhibit cell proliferation. For both untransfected Huh7 cells and those transfected with the control shRNA (Huh7-shControl), doxorubicin completely blocked their proliferation at a concentration between 50 and 150 nM, which was also the concentration that resulted in maximal cleavage of CREB3L1. For Huh7-shCREB3L1 cells, doxorubicin at concentrations up to 500 nM failed to block their proliferation. These concentrations of doxorubicin were not enough to trigger apoptosis of Huh7 cells, which became apparent only when the cells were treated with 5 μM of the compound.

If proteolytic activation of CREB3L1 is required for doxorubicin to inhibit cell proliferation, then the amount of CREB3L1 expressed in cancer cells may determine their sensitivity to doxorubicin. To test this hypothesis, we analyzed SV589 cells, an immortalized line of human fibroblasts (12), and MCF-7 cells, a line of human breast cancer cells (13). Compared to Huh7 cells, expression of CREB3L1 was higher in SV589 cells and lower in MCF-7 cells. The sensitivity of the cells to growth inhibition by doxorubicin followed the order of CREB3L1 expression Similar to Huh7 cells, knockdown of CREB3L1 by two duplexes of siRNA targeting different regions of CREB3L1 in SV589 cells made them more resistant to doxorubicin. Since MCF-7 cells expressed very little CREB3L1, we used these cells to study the effect of CREB3L1 overexpression on sensitivity to doxorubicin. We stably transfected MCF-7 cells with a plasmid encoding CREB3L1 and selected one clone of the cells with relatively low expression (MCF7/pCREB3L1(L); 8-fold above parental cells) and another clone with high expression of CREB3L1 (MCF7/pCREB3L1(H); 300-fold above parental cells). The 8-fold overexpression of CREB3L1 in MCF7/pCREB3L1(L) cells lowered the IC₅₀ for doxorubicin from 500 nM to 10 nM, and the 300-fold overexpression of CREB3L1 in MCF7/pCREB3L1(H) cells further reduced the IC₅₀ to ˜1 nM. In this experiment, cells were treated with doxorubicin for 2 days. To determine the effect of CREB3L1 expression on proliferation of the cells treated with doxorubicin for a longer period of time, we incubated MCF-7 and MCF7/pCREB3L1(H) cells with 15 nM doxorubicin for 6 days. This treatment did not affect proliferation of MCF-7 cells, but markedly blocked proliferation of MCF7/pCREB3L1(H) cells, as determined by direct cell counting and by measurement of cellular DNA content. Thus, CREB3L1 expression level is a key determinant of cellular sensitivity to doxorubicin.

We then determined the relationship between doxorubicin-induced cleavage of CREB3L1 and DNA breaks caused by inhibition of topoisomerase. Doxorubicin induced appearance of histone γH2AX, a marker for DNA breaks. However, this effect was unaffected by knockdown of CREB3L1 expression. This result indicates that cleavage of CREB3L1 does not lead to doxorubicin-induced DNA breaks. To investigate whether DNA breaks may lead to cleavage of CREB3L1, we examined etoposide, another chemotherapeutic drug that inhibits topoisomerase (14). Unlike doxorubicin, etoposide failed to induce cleavage of CREB3L1, even though the compound was as effective as doxorubicin in causing DNA breaks. Accordingly, knockdown of CREB3L1 in Huh7 cells did not increase their resistance to etoposide, and overexpression of CREB3L1 in MCF7 cells also did not increase their sensitivity to the compound. These results indicate that induction of CREB3L1 cleavage by doxorubicin is not related to its activity to inhibit topoisomerase. Besides etoposide, CREB3L1 is also not required for bleomycin or paclitaxel to inhibit cell growth, an observation indicating that CREB3L1 is specifically involved in doxorubicin-induced suppression of cell proliferation.

RIP of membrane-bound transcription factors is known to be a signal transduction pathway that transfers signals from the ER to nucleus (5). Since ER is the site for synthesis of most lipids, we wondered whether doxorubicin may alter homeostasis of certain lipids that may result in cleavage of CREB3L1. Daunorubicin, a chemotherapeutic drug derived from doxorubicin, was reported to induce de novo synthesis of ceramide (15). We determined that doxorubicin also stimulated ceramide synthesis by showing that treatment with the compound increased the amount of tritium-labeled palmitate incorporated into ceramide in Huh7 cells. Myriocin, an inhibitor of ceramide synthesis (16), inhibited doxorubicin-induced cleavage of CREB3L1 in a dose-dependent manner. Co-treatment with myriocin also rendered the cells more resistant to doxorubicin. Exogenously added C₆-ceramide, a cell-permeable analogue of ceramide that contains a short acyl chain (C₆-ceramide), stimulated CREB3L1 cleavage even in the absence of doxorubicin. These results indicate that doxorubicin-induced synthesis of ceramide leads to cleavage of CREB3L1. Thus, CREB3L1 suppresses cell proliferation in response to accumulation of ceramide. This conclusion was further supported by the observation that knockdown of CREB3L1 in Huh7 cells completely abolished the ability of C₆-ceramide to inhibit cell proliferation.

The current study establishes a crucial role for CREB3L1 in mediating inhibition of cell proliferation in response to doxorubicin-induced synthesis of ceramide. The concentration of doxorubicin required to proteolytically activate CREB3L1 is within clinically relevant concentration ranges found in the serum of patients treated with the drug (<1 μM) (1). These findings indicate that the clinical response to doxorubicin is determined by the level of CREB3L1 produced in tumor cells. Thus, measuring CREB3L1 expression in tumor cells is useful in identifying cancer patients who are most likely to benefit from doxorubicin treatment.

For our early data a rabbit polyclonal antibody against human CREB3L1 was generated by immunizing rabbits with a protein consisting of the NH₂-terminal 290 amino acid residues of human CREB3L1. We also raised monoclonal antibodies with this same immunogen. However, we subsequently sought to develop antibodies with improved CREB3L1 binding specificity and less cross-reactivity. These efforts proved challenging, as it was not obvious how to generate a better antibody—there was poor overlap between relatively unique regions, with reference to human protein databases, and regions identified by antigenic prediction algorithms Furthermore, it was apparent that many others had failed to do so, as we tested many purported CREB3L1 antibodies from third parties, and none provided improved specificity.

However, by creatively examining for unique regions we eventually developed antibodies directed to epitopes within amino acids 7-41 of CREB3L1 with improved CREB3L1 specificity and less cross-reactivity, and improved performance and suitability for immunocytochemistry of tumor samples. The particular suitability of this region was unexpected because it was not predicted as a good antigen because of the lack of charged residues.

In exemplary methods we immunized mice with a synthetic peptide corresponding to amino acids 7-41 of CREB3L1 conjugated with KLH. The monoclonal antibody was then produced according to standard procedures. The initial screen was performed with ELISA against the peptide. The positive clones were then screened by western blot with lysates of Huh-7 cells known to express CREB3L1, with the cells stably transfected with a shRNA targeting CREB3L1 as a negative control. Clones that were positive in recognizing endogenous CREB3L1 were selected for single-cell cloning. The resultant 10H1 antibody is not only very sensitive but also extremely specific in detecting endogenous CREB3L1 in multiple cell lines by western blot. This antibody is also capable of detecting CREB3L1 cleavage in xenograft tumors implanted in mice after doxorubicin treatment by western blot. This antibody works well with IHC and provides a useful diagnostic tool to identify patients more likely to be benefited from doxorubicin treatment, particularly high dosage treatments.

We have also used xenograft mice model to show that CREB3L1 expression in renal cancers directly taken from human patients determines their response to doxorubicin. In exemplary xenograft methods we first screened ˜30 human kidney tumor samples directly taken from patients, and found 3 of which expressed CREB3L1. We then implanted tumors either expressing or not expressing CREB3L1 into mice, and treated them with doxorubicin with a dose that was about 7 times lower than that applied to human clinically. The results showed while the tumors without CREB3L1 expression did not respond to doxorubicin treatment, the drug had a profoundly inhibited those expressing CREB3L1, and the effect was better than rapamycin, the current standard treatment for renal cancer.

We also validated our antibodies using immunohistochemistry for human tumors and for screening of lymphoma. In exemplary studies kidney tumors without CREB3L1 expression as determined by 10H1 staining presented much less doxorubicin sensitivity than kidney tumors staining for CREB3L1 expression, measured at day 3, 6, 9, 12, 15, 18 and 21 time points.

### REFERENCES

1. D. A. Gewirtz, A critical evaluation of the mechanisms of action proposed for the antitumor effects of the anthracycline antibiotics adriamycin and daunorubicin. Biochem. Pharmacol. 57, 727 (1999).

2. V. Lee, D. Harris, Adriamycin nephropathy: A model of focal segmental glomerulosclerosis. Nephrology 16, 30 (2011).

3. B. Denard et al., The membrane-bound transcription factor CREB3L1 is activated in response to virus infection to inhibit proliferation of virus-infected cells. Cell Host & Microbe 10, 65 (2011).

4. Y. Omori et al., OASIS is a transcriptional activator of CREB/ATF family with a transmembrane domain Biochem. Biophys. Res. Commun. 293, 470 (2002).

5. M. S. Brown, J. Ye, R. B. Rawson, J. L. Goldstein, Regulated intramembrane proteolysis: A control mechanism conserved from bacteria to humans. Cell 100, 391 (2000).

6. H. Nakabayashi, K. Taketa, K. Miyano, T. Yamane, J. Sato, Growth of human hepatoma cell lines with differentiated functions in chemically defined medium. Cancer Res 42, 3858 (1982).

7. R. B. Rawson, D. Cheng, M. S. Brown, J. L. Goldstein, Isolation of cholesterol-requiring mutant Chinese hamster ovary cells with defects in cleavage of sterol regulatory element-binding proteins at Site 1. J. Biol. Chem. 273, 28261 (1998).

8. R. B. Rawson et al., Complementation cloning of S2P, a gene encoding a putative metalloprotease required for intramembrane cleavage of SREBPs. Mol. Cell 1, 47 (1997).

9. J. Ye et al., ER stress induces cleavage of membrane-bound ATF6 by the same proteases that process SREBPs. Mol. Cell 6, 1355 (2000).

10. T. Murakami et al., Signalling mediated by the endoplasmic reticulum stress transducer OASIS is involved in bone formation. Nat Cell Biol 11, 1205 (2009).

11. C. J. Sherr, J. M. Roberts, CDK inhibitors: positive and negative regulators of G1-phase progression. Genes & Dev. 13, 1501 (1999).

12. T. Yamamoto et al., The human LDL receptor: A cysteine-rich protein with multiple Alu sequences in its mRNA. Cell 39, 27 (1984).

13. H. D. Soule, J. Vazguez, A. Long, S. Albert, M. Brennan, A human cell line from a pleural effusion derived from a breast carcinoma. J. Natl. Cancer Inst. 51, 1409 (1973).

14. H. F. Stähelin, A. von Wartburg, The chemical and biological route from podophyllotoxin glucoside to etoposide: Ninth Cain Memorial Award Lecture. Cancer Res. 51, 5 (1991).

15. R. Bose et al., Ceramide synthase mediates daunorubicin-induced apoptosis: An alternative mechanism for generating death signals. Cell 82, 405 (1995).

16. Y. Miyake, Y. Kozutsumi, S. Nakamura, T. Fujita, T. Kawasaki, Serine palmitoyltransferase is the primary target of a sphingosine-like immunosuppressant, ISP-1/myriocin. Biochem. Biophys. Res. Commun. 211, 396 (1995).

17. J. E. Metherall, J. L. Goldstein, K. L. Luskey, M. S. Brown, Loss of transcriptional repression of three sterol-regulated genes in mutant hamster cells. J. Biol. Chem. 264, 15634 (1989).

18. J. Sakai et al., Sterol-regulated release of SREBP-2 from cell membranes requires two sequential cleavages, one within a transmembrane segment. Cell 85, 1037 (1996).

19. G. Liang et al., Diminished hepatic response to fasting/refeeding and Liver X Receptor agonists in mice with selective deficiency of Sterol Regulatory Element-binding Protein-1c. J. Biol. Chem. 277, 9520 (2002).

20. C. M. Adams et al., Cholesterol and 25-hydroxycholesterol inhibit activation of SREBPs by different mechanisms, both involving SCAP and Insigs. J. Biol. Chem. 279, 52772 (2004).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

What is claimed is:

1. A method of detecting CREB3L1 expression of cancer cells of a patient, the method comprising the step(s) of:
immunodetecting CREB3L1 expression of the cells with an antibody that specifically binds an epitope within residues 7-41 of CREB3L1.

2. The method of claim 1 wherein the method doses doxorubicin chemotherapy for the cancer patient, wherein the expression indicates suitability for the patient of high dosage doxorubicin chemotherapy, as opposed to a conventional lower standard dosage that would otherwise be indicated.

3. The method of claim 1 further comprising the step:
isolating or obtaining the cells as a biopsy from the patient.

4. The method of claim 1 further comprising the steps:
translating the CREB3L1 expression to an indication of doxorubicin chemotherapy suitability for the patient; and
recording the indication.

5. The method of claim 1 wherein the method further comprises the step(s) of:
(i) providing for the patient information indicating said suitability of a CREB3L1-mediated chemotherapy;
(ii) providing for the patient a prescription for the CREB3L1-mediated chemotherapy; and/or
(iii) administering to the patient the CREB3L1-mediated chemotherapy.

6. The method of claim 5 consisting of or consisting essentially of the recited steps.

7. The method of claim 1 wherein the method is a drug screen and comprises determining a candidate anti-cancer drug activates CREB3L1, and determining that the drug inhibits growth of a CREB3L1 expressing cancer cell.

8. The method of claim 1 comprising immunohistochemistry (IHC) detection of the CREB3L1 in a tumor sample.

9. The method of claim 1 wherein the antibody is a monoclonal antibody.

10. The method of claim 1 wherein the antibody is a mouse monoclonal antibody.

11. The method of claim 1 further comprising the step of:
treating the patient with doxorubicin chemotherapy.

12. The method of claim 11 wherein the method doses doxorubicin chemotherapy for the cancer patient, wherein the expression indicates suitability for the patient of high dosage doxorubicin chemotherapy, as opposed to a conventional lower standard dosage that would otherwise be indicated.

13. The method of claim 11 further comprising the step:
isolating or obtaining the cells as a biopsy from the patient.

14. The method of claim 11 further comprising the steps:
translating the CREB3L1 expression to an indication of doxorubicin chemotherapy suitability for the patient; and
recording the indication.

15. The method of claim 11 wherein the method further comprises the step(s) of:
(i) providing for the patient information indicating said suitability of a CREB3L1-mediated chemotherapy;
(ii) providing for the patient a prescription for the CREB3L1-mediated chemotherapy; and/or
(iii) administering to the patient the CREB3L1-mediated chemotherapy.

16. The method of claim 11 consisting of or consisting essentially of the recited steps.

17. The method of claim 11 wherein the treating provides high dosage doxorubicin chemotherapy, as opposed to a conventional lower standard dosage that would indicated in the absence of the immunodetecting step.

18. The method of claim 11 wherein the antibody is a monoclonal antibody.

19. The method of claim 11 wherein the antibody is a mouse monoclonal antibody.

20. An isolated antibody that specifically binds an epitope within residues 7-41 of CREB3L1, or a monoclonal antibody that specifically binds an epitope within residues 7-41 of CREB3L1.
